(19) United States
(12) Patent Application Publication
(10) Pub. No.: US 2005/0120027 A1
(43) Pub. Date: Jun. 2, 2005

(54) Method and device for presenting, managing and exploiting graphical queries in data management systems

(75) Inventor: Herman Jan Ter Horst, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics N.V., NL-5621 BA Eindhoven (NL)

(21) Appl. No.: 10/499,623

(22) PCT Filed: Dec. 10, 2002
(86) PCT No.: PCT/IB02/05318

(30) Foreign Application Priority Data
Dec. 24, 2001 (EP) 01205084.5

(51) Int. Cl. G06F 7/00; G06F 17/00; G06F 19/00; G01N 33/48; G01N 33/50

(52) U.S. Cl. 707/100; 702/19

(57) ABSTRACT

The invention concerns a method and device for presenting, managing and exploiting graphical queries in data management systems. In the method queries in data management systems may be handled graphically in a simple fashion to form a query formalism incorporating objects and their relationships, and including specialization relationships between types of objects. The method comprises defining in a graphical format a semantic model defining the structure of data within a database and using said semantic model to define a database query, the method being characterised in that the semantic model comprises a type graph comprising types of objects, and attributes and specializations linking types to one another to define relationships between said types.

The invention relates to a method and device for presenting, managing and exploiting graphical queries in data management systems.

In particular, the invention concerns a method in which queries in data management systems may be presented, managed and exploited graphically in a simple fashion to form a query formalism incorporating objects and their relationships, and including specialization relationships between types of objects. Here data management systems include the well-known database management systems, and also include systems for managing data on the Web, for example.

Some database management systems available at present include within them the possibility for portraying a query graphically. Such graphical queries give a pictorial depiction of the objectives of a search and facilitate users in the construction of queries as they avoid the need for user knowledge of special query languages such as SQL.

The Access data base system from Microsoft is based on the relational model and it has long been recognised that information modellers cannot express enough semantics using the relational model. In particular, objects and specialization relationships do not appear. It is also a problem to arrive at clear principles for organizing graphical queries in the presence of objects and specialization relationships.

The graphical user interface of Access may be utilised to develop an updateable view, in the form of an interactive, form-based data based application. However, in many cases, when one constructs a form with nested sub-forms, one needs to develop and supply a large number of graphical queries. A similar remark applies to the construction of textual reports by means of the user interface of Access.

Another notable feature of the Access system is that the query user interface does not make full use of the predefined relationships that may exist between tables of the database. Typically therefore, when one graphically constructs queries, it is necessary to repeat the specification of such predefined information on a frequent basis.

It is an aim of embodiments of the present invention to provide a means by which a graphical representation of queries can be given for specifying graph-based queries, based on objects, relationships between objects, and specialization relationships between types of objects.

It is another aim to provide a means by which possibly complex, form-based, interactive database applications may be developed using graphical queries.

It is another aim to provide a means by which possibly complex, textual reports may be developed using graphical queries.

A further aim of embodiments of the invention is to provide a means by which the construction of graphical queries is facilitated.

According to an aspect of the invention, there is provided a graphical user interface for a data management system, the user interface comprising:

means for defining in a graphical format a semantic model defining the structure of data within a database; and means for using said semantic model to define a database query, the interface being characterised in that the semantic model comprises a type graph comprising types of objects, and attributes and specializations linking types to one another to define relationships between said types.

According to a second aspect of the invention, there is provided a method of presenting and managing graphical queries in a data management system comprising: defining in a graphical format a semantic model defining the structure of data within a database; and using said semantic model to define a database query, the method being characterised in that the semantic model comprises a type graph comprising types of objects, and attributes and specializations linking types to one another to define relationships between said types.

Said type graph is preferably represented according to a pre-defined graphical notation which may comprise types being represented in a particular manner and associated with a type name and attributes and specializations being represented as linking types to one another.

Using said semantic model may comprise using said type graph to define a query graph.

Said query graph preferably comprises query types, query attributes and query specializations being respectively derived from types, attributes and specializations of said type graph.

A query type is preferably associable with a selection and/or a negation.

Preferably, a query graph is constructable by means of copy and paste operations carried out on a type graph.

A query graph may further comprise a reference to one or more other queries.

An interactive, form-based application, which possibly contains sub-forms, may be realised by construction of a query graph.

Modification of data may be achieved utilising the form-based application.

A textual report which may contain nested sub-reports of various different, regular structures, may also be realized by construction of a query graph.

For a better understanding of the invention, and to show how embodiments of the same may be carried into effect, reference will now be made, by way of example, to the accompanying diagrammatic drawings in which:

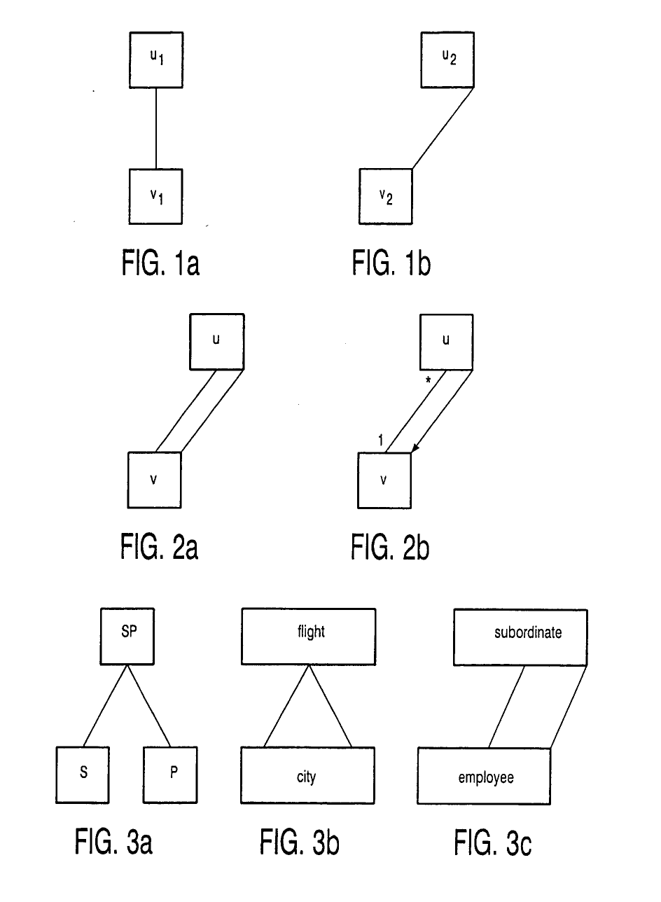

FIGS. 1A and 1B illustrate a graphical notation for semantic models;

FIG. 2 describes the equivalent UML notation;

FIGS. 3A-C show three example semantic models;

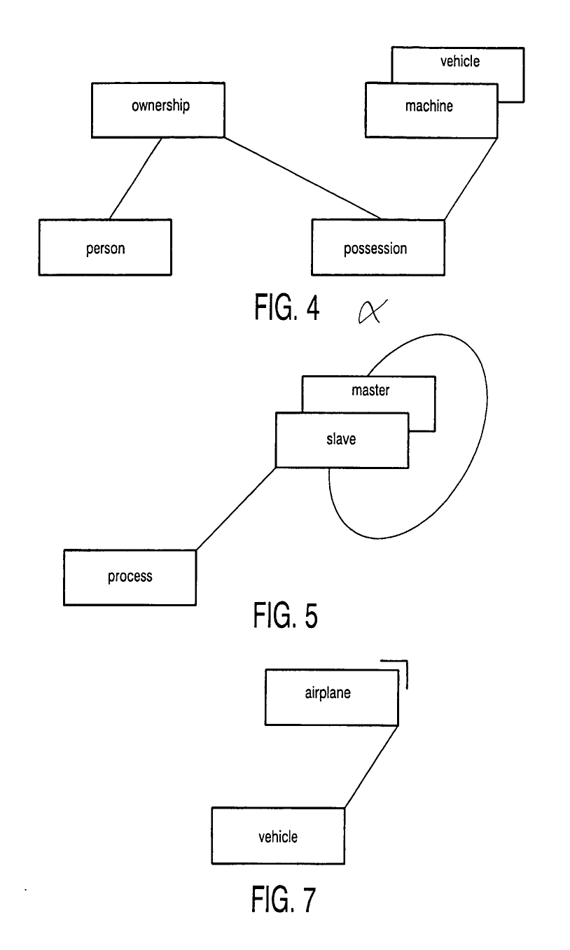

FIG. 4 is a semantic model illustrating a situation in which ownership of articles of various types is present;

FIG. 5 illustrates a semantic model for information about software processes;

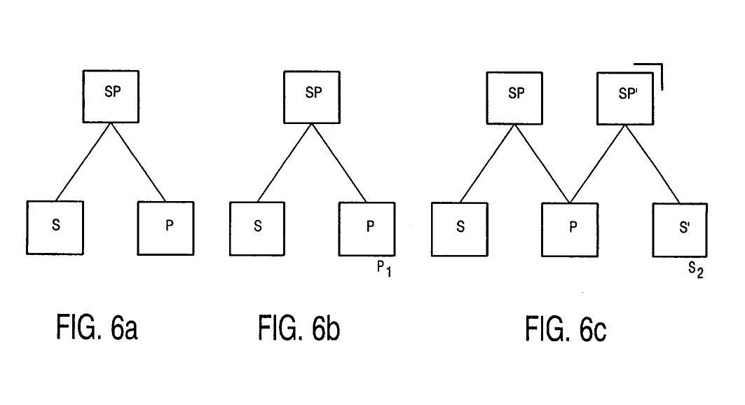

FIGS. 6(A)-(C) illustrates how a graph-based query may be constructed based upon a semantic model type graph;

FIG. 7 illustrates format of a query concerning aeroplanes;

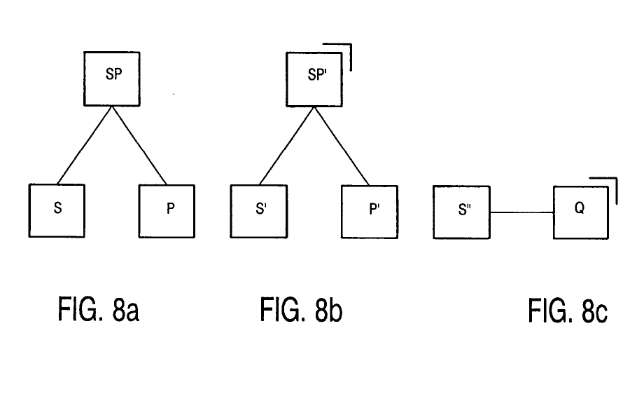

FIGS. 8(A)-(C) illustrates the construction of a composite query based upon a semantic model type graph;

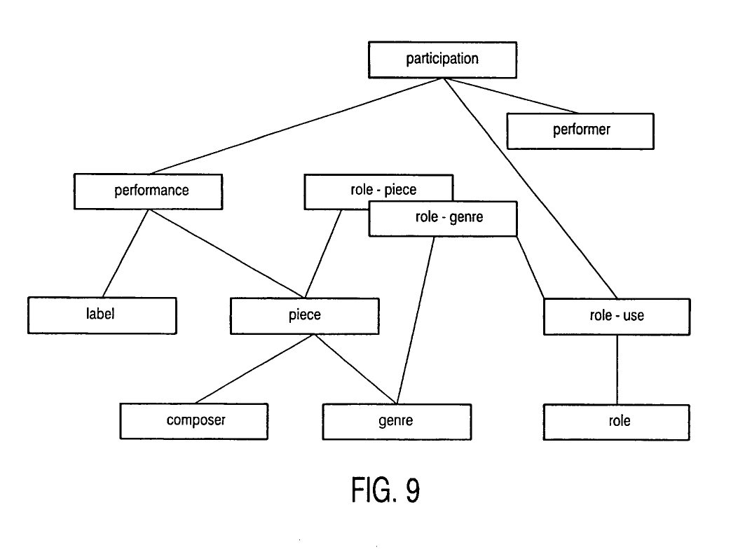

FIG. 9 is an example of a more complicated semantic model, involving music performances and performers; the next two figures use this example to show how interactive inspection and modification of information may be achieved;

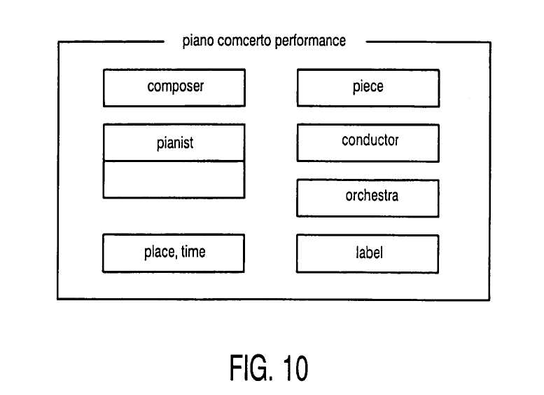

FIG. 10 is an example of a form-based application; and

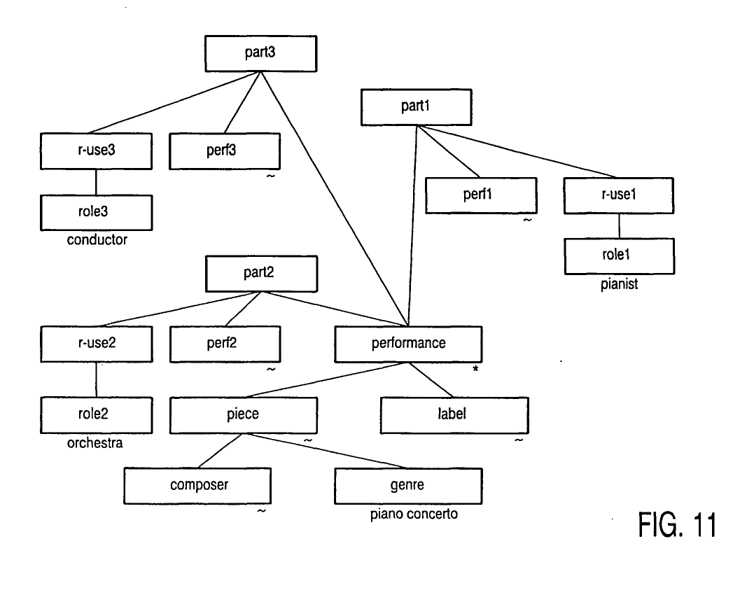

FIG. 11 illustrates a complex graph-based query.

The present invention provides a means by which a data modelling and retrieval formalism can be specified that preserves certain strengths of the well-known and widely used relational model [Codd 1970] [Codd 1982], and removes some of its limitations. Limitations of the relational model involve such fundamental issues as semantics (objects), user interfaces, and updatable views. Strengths of the relational model which it is wished to preserve include: data independence, view independence, formality, and simplicity.

Data independence ensures that information modellers do not need to think about physical representation details. The term view independence expresses the fundamental achievement that data can be defined independently of views, and that views can be obtained in an easy, non-procedural way. In some current relational arrangements, graphical user interfaces make the creation of views (queries) easier than by using query languages such as SQL. Together, data independence and view independence realise the standard three-level data base architecture [Tsichritsis and Klug 1978]. The formality of the relational model is also important: it provides a clear and precise mathematical foundation, giving concepts a clear meaning and providing a cornerstone for software development. The simplicity of the relational model is given by its basic primitives: tuples, with values in certain domains, and forming sets called tables or relations.

As to the semantics of data to be stored and retrieved, the relational model is limited. A relational table describes a relationship between certain attributes, while the relationships between different tables are considered separately, using constraints (dependencies). No objects or specialization relationships between types of objects are incorporated. There is no widely-used, mathematically-founded data modelling formalism which allows information modellers to express more semantics than with the relational model. There has, however, been much interest in object-oriented approaches. An important perspective offered by the use of explicit objects is the stronger support for data semantics.

Graphical query user interfaces which are currently available are, at present, simply provided as a means by which a user can construct queries which are interpreted into a language such as SQL, without the user needing to have a knowledge of SQL. Therefore, graphical user interfaces currently available provide a graphic interface that is interpreted internally in terms of an earlier, textual interface or formalism. It is a problem to arrive at clear principles for organizing graphical queries in the presence of objects and specialization relationships.

As to updatable views, it would be desirable to have a comprehensive, global treatment of updatability of information, via an interactive form-based application that presents the information in some alternative view. In the relational context, such a treatment is difficult to develop, since the possibility of this kind of updatability generally depends on relationships between tables, which are not formalized as part of schemas.

For a proper understanding of the concepts of the present invention, it is necessary to understand a number of modelling concepts that are used in its explanation. In the description that follows, we use the term semantic model for what is often called schema. In the sub-sections that follow, semantic models and their instances are described, there is discussed a convenient graphical notation for semantic models and examples are given. Queries, updatable views (form-based applications), and textual reports are then discussed with examples.

Semantic models, object-oriented instances

A key characteristic of semantic models is that they are defined using just one type concept. An instance of a semantic model contains objects. Types are related using attributes and specializations, which are connections from one type to another type. In this way, the type graph of a semantic model is formed. It is convenient to refer to either attributes or specializations as arrows. The arrows of a type are the outgoing arrows of the type.

Specializations indicate specialization relationships between types, and are used to model diversity in data structure. For instance, if e is a specialization from the type v to the type w, then v is a direct specialization of w. In this case, each instance of v is also an instance of w, and conversely, each instance of w may also be an instance of v. In this way, each object of an instance of a semantic model is an instance of one or more types of the model. Pairs of specializations of a type can be specified as being disjoint (i.e. as sharing no instances).

All relationships between objects are defined in terms of attributes, which are essentially n:1-relationships (that is, functions, or inverses of 1:n-relationships). If e is an attribute from the type v to the type w then each instance of v is related to (we shall say refers to) a unique instance of w in respect of the attribute e; conversely, each instance of w is related to a number of (that is, one of 0, 1, 2, . . . ) instances of v. In this way, the outgoing attributes of a type can be viewed as single-valued, and the incoming attributes can be viewed as set-valued.

A scalar type in a semantic model is a type without (outgoing) arrows, or a direct specialization of a scalar type, where the direct specialization has no (outgoing) attributes. For each scalar type b, a domain of values needs to be defined, and each instance of b has a value from this domain. If there is a specialization from a scalar type v to a scalar type w, and if an object x is an instance of v and w, then the value of x should be the same for v and w. A scalar attribute is an attribute pointing to a scalar type. If a type is not scalar, it is called composite. It should be noted that scalar types may range from simple types such as integers and strings, to complicated types involving graphics or multimedia. Scalar types do not need to be generic, but may also be used to express special constraints, as in the scalar type “Integer with values between 0 and 100”.

If a type v has more than one attribute of (i.e., pointing to) the same type w, these attributes should be distinguished using roles. For example, a type “person” might have two attributes of type “phone number” one of role “home” and one of role “work”. In this way, roles may be needed to distinguish the meaning of attributes. It is natural to impose a constraint on semantic models called convertibility, which requires distinct types to have distinct definitions (that is, attributes and roles). One may add static constraints to a semantic model, making assertions in terms of the instances of the types, and restricting the possible instances of the model.

The definition of semantic models and object-oriented instances just given can be mathematically formalized, in terms of sets, functions and graphs. It should be noted that inheritance is obtained without imposing restrictions on the structure of type graphs; it may be assumed, however, without loss of generality, that the specializations in a type graph cannot be combined to form a cyclic path.

Graphical notation

The invention does not depend on the precise details of the graphical notation used for representing semantic models and graphical queries. However, some sort of notation needs to be used in a graphical user interface. In this document, we use a convenient graphical notation that facilitates the understanding of the global aspects of semantic models. This notation is based on a notation devised by Ter Bekke [Ter Bekke 1992]. In this notation, types are depicted as rectangles containing their name. Scalar types that are not essential to the global understanding of a semantic model are not drawn. In this notation, it is not necessary to draw arrows (that is, attributes or specializations) with arrow heads: an arrow from a type v to w is drawn as a connection from the lower side of v to the upper side of w. An attribute is drawn as a middle-middle connection, and a specialization as a corner-corner connection, as is illustrated in FIG. 1. By suitably positioning the types, as many of the arrows as possible are drawn as downward connections. Disjoint specializations of a type are drawn using overlapping rectangles. When one observes a drawing of a semantic model in the downward direction, one can read definite assertions on the instance level: a downward middle-middle connections can be read as “is related to a unique”, and a downward corner-corner connections can be read as “is a”. In the upward direction, one can read indefinite assertions: an upward middle-middle connection can be read as “is related to a number of”, and an upward corner-corner connection can be read as “may be a”. In this way, the drawing rules lead to a great deal of transparency.

For instance, referring to FIG. 1(a) the notation denotes that each instance of the type u₁ is related to a unique instance of the type v₁ and each instance of v₁ is related to an arbitrary number of instances of u₁. FIG. 1(a), as it is a middle-middle connection concerns attributes.

In FIG. 1(b) as the arrow is a corner-corner connection, the relationship is illustrated as a specialization relationship. It may be read by saying that each instance of the type u₂ is also an instance of the type v₂ and each instance of v₂ may also be an instance of u₂.

It is possible to extend this notion in a simple way, in order to obtain an equivalent in the form of the well-known and widely used UML notation [Booch et al. 1999]. This is illustrated with an example in FIG. 2. FIG. 2(a) depicts a semantic model with two types u and v, and with two arrows from u to v, an attribute and a specialization. By extending the attribute with cardinality information, and by including an open triangle as the endpoint of the specialization, we get UML notation: see FIG. 2(b). In this way, the graphical notation used here can be viewed as a (simplification of a) small subset of UML, providing transparent, global overviews of type graphs by means of a convenient layout convention.

FIG. 3 gives some concrete examples of semantic models. FIG. 3(a) presents a semantic model for suppliers (S) and parts (P). A third type (SP) is used, with attributes pointing to both types S and P. Each instance of SP represents a combination of a part and a supplier supplying this part. In addition, there is a scalar type “name” used for both suppliers and parts, which is not shown in FIG. 3(A). Reading FIG. 3(a) it may be noted that each instance SP is related to a unique supplier and to a unique part. It is also noted that each supplier is related to a number of combinations SP and each part is related to a number of combinations SP. This gives an example of an m:n relationship: each instance of S can be related via the type SP to multiple instances of P, and each instance of P can be related to multiple instances of S.

FIG. 3(b) presents a semantic model for aeroplane flights. Each flight is described by two cities, a source and a destination. Thereby, each flight is related to a unique source city, and is related to a unique destination city. Each city may be related to a number of flights. The roles “source” and “destination” of the two attributes from the type flight to the type city are not displayed in the Figure.

FIG. 3(c) presents a semantic model for simple organisations. In this model, there are employees, most of whom are also subordinates. There are two arrows from the types subordinate to the type employee: a specialization, indicating that each subordinate is an employee, and an attribute, indicating the employee who is directly superior to a subordinate.

As another illustration of the use of specializations, consider a situation in which people may own and possibly share ownership of articles of several types (for example, machines, vehicles etc.). In a relational implementation, one would typically introduce an m:n relationship for each kind of ownership. However, it is more attractive to introduce just two things: an additional type “possession” of which the earlier types are specializations, in combination with one “ownership” type. See FIG. 4. From FIG. 4 it can be seen that ownership is related to a unique person. Ownership is also related to a unique possession. The machine is a possession, a vehicle is a possession etc. Each possession may be related to a number of ownerships.

In the example of FIG. 5, there is shown a semantic model for information about software processes, in which there is a type process having two disjoint specializations “master” and “slave”. Using an attribute, each slave process is assigned to be secondary to a certain master process.

Null values and m:n relationships

In the context of the relational model, it is often customary to allow attributes to be ‘optional’, that is, to allow attributes to have a null value. It should be noted that in the context of semantic models and object-oriented instances this can be realized by means of specializations. For example, a type with 4 optional attributes can be described by adding 4 additional types which are direct specializations of the given type; these specialization types are given the desired attributes. There is no need to define a special type for each combination of the specialization types that might be used: we do not make the assumption that there is an “intersection type” for each pair of types that share instances. By making systematic use of specializations in this way, there is no need to allow null values (of references of objects to other objects).

If e is an attribute from the type v to the type w, and if the object x is an instance of v, then x is related to a a certain number n of instances of w, in respect of the attribute e: we assumed above that n is always 1. However, the findings of the invention related to graphical queries, user interfaces, form-based applications and textual reports do not depend on the assumption that n=1. In the preceding paragraph we saw that it is not necessary to allow n=0, since specializations can be used instead of null values. In addition, it is not necessary to allow the possibility that n>1: an m:n relationship between two types u and v can be realized by means of an additional type w in combination with two attributes (n:1 relationships), one from w to u and one from w to v (compare the example of FIG. 3). It is possible to introduce optional attributes (null values) and m:n relationships as additional primitives. An optional attribute is a directed connection from one type to another type, and an m:n relationship is an undirected connection between two types. These constructs can be realized in terms of the original primitives in the ways just described, by means of a specialization and two attributes, respectively. When one realizes m:n relationships as an additional primitive in this way, it is natural to maintain the situation that there is exactly one “connecting object” that is an instance of the type w just mentioned, for each accordingly related pair consisting of an object that is an instance of u and an object that is an instance of v. The graphical notation for semantic models could be extended by drawing an optional attribute in a dashed way, and by drawing an m:n relationship as a middle-middle connection between vertical sides.

Relational realization of semantic models and object-oriented instances

We describe two ways to realize semantic models and object-oriented instances as described above. First we present a realization on the basis of the relational model, then a realization in terms of Web data in the form of XML.

In the relational realization, a table is introduced for each type of a given semantic model; this table (only) contains a unique tuple for each object that is an instance of the type. The main idea is that an arrow in the semantic model, from the type v to a type w, can be implemented using a relational attribute in the table for v, which is a foreign key for the table for w. If ‘global object identifiers’ are used, it is not necessary to do this for all arrows, but it is sufficient to do this for all semantic attributes. (In this section, we call an attribute of a semantic model a semantic attribute, for clarity, in order to distinguish it from a relational attribute.)

To summarize, a semantic model can be realized using a relational schema defining a table Tᵥ for each type v of the semantic model, with the following relational attributes:
a key attribute kᵥ for uniquely identifying the objects that are an instance of v, so that the set of tuples in Tᵥ can be identified with the set of objects that are an instance of the type v;
if v is a scalar type, an attribute describing the value of each object (the domain of this attribute is the domain of the scalar type);
for each semantic attribute of v, from v to a certain type w, an attribute whose value, for a tuple in Tᵥ describing an object x that is an instance of v, is the key value, in the table T_{w}, of the object referred to by x.

These attributes suffice when key attribute values are chosen as global object identifiers: that is, when for each pair of types v,w of the semantic model, and for each object x that is an instance of both v and w, we have kᵥ(x)=k_{w}(x), and when, for each pair of distinct objects x,y we have kᵥ(x)≢k_{w}(x) for each type v such that x is an instance of v and for each type w such that y is an instance of w. If the key attribute values are not chosen as global object identifiers, then it is sufficient to add the following attributes to the table Tᵥ:
for each specialization from v to a type w, an attribute with value k_{w}(x) for each object x that is an instance of v.

It is clear that, for each semantic attribute from a type v to a type w, the relational attribute included in Tᵥ is a foreign key for table T_{w}. In other words, a 1:n referential integrity constraint is defined between tables Tᵥ and T_{w}. When key attribute values are not used as global object identifiers, there is a stronger constraint on the relational attributes used for the specializations: for each relational attribute implementing a specialization from a type v to a type w, and for each tuple in the table T_{w}, there should be at most one related tuple in Tᵥ. This stronger constraint can be realized when using the Access system, for example: by introducing key attribute values independently for different tables (that is, no global object identifiers), a specialization from the type v to the type w can be implemented by using a 1:1 referential integrity constraint from table Tᵥ to table T_{w}.

It is clear that the definition of semantic models and object-oriented instances implies that several further static constraints should be added to the relational schema just described. In an alternative relational realization, one might incorporate a type together with certain specializations of this type into one relational table, by allowing null values for the semantic attributes of these specializations.

The relational realization of semantic models and object-oriented instances just discussed makes clear that the notion of semantic model can be applied to a relational database management system that incorporates facilities for referential integrity constraints. In this interpretation, a type should be viewed as a relational table, an attribute should be viewed as a 1:n referential integrity constraint, and a specialization should be viewed as a 1:1 referential integrity constraint. Hence a relational schema, together with referential integrity constraints, may be interpreted in terms of a semantic model. As to instances, objects should be viewed in this interpretation as the entities described by tuples in relational database tables. In this way, several findings of the invention are not only relevant in an object-oriented framework as considered here, but also pertain to relational database management systems. This includes in particular the findings related to graphical queries, user interfaces, form-based applications, and textual reports. The descriptions given below are phrased in terms of objects and relationships (references), but can be rephrased in the relational context by means of our relational realization.

XML realization

The language XML has emerged as a standard for exchange of data on the World Wide Web. We describe an XML realization of general semantic models and object-oriented instances. This XML realization is analogous to the relational realization of the preceding section. Just as in the preceding section, we call an attribute of a semantic model a semantic attribute; an attribute in XML will be called an XML attribute.

Given a semantic model, the purpose is to represent instances of this model in the form of XML documents of a certain structure. We introduce an XML element type for each type of the semantic model. For each type v of the semantic model, the root of the XML element tree contains a unique element for each object that is an instance of v. If an object x is an instance of more than one type, then several elements will be introduced in this way for x. Each XML realization of an instance necessarily carries irrelevant ordering information. For example, the sequence in which the elements describing the objects appear is arbitrary. If we enumerate the set of types, and also the element types for these types, as v₁, . . . , vₙ, then the content type of the root element could, for example, be defined in XML as v₁* . . . vₙ* or (v₁| . . . |vₙ)*.

In a realization with global object identifiers, the element type declaration for a type v includes the following XML attributes:
an object identification attribute for uniquely characterizing objects,
a scalar value attribute if v is a scalar type,
for each semantic attribute from v to a type w, a reference attribute with values being the appropriate object identification attributes of objects referred to.

In this XML realization, with global object identifiers, it is not possible to use XML's ID attributes since XML requires that ID values uniquely identify the elements that bear them. ID and IDREF attributes can be used, however, in an alternative version, without global object identifiers, of the attributes of the element type for a type v of the semantic model (this is analogous to the relational realization without global object identifiers already described).

Graph-based queries

Given the understanding of semantic models explained above, it is now possible to turn to the formulation of a graph-based query which could be adopted for use in graphical user interfaces of the type, for instance, offered by Microsoft Access.

A basic query is defined in terms of a query graph, consisting of query types and query arrows. Each query type or query arrow is a copy of a certain type or arrow, respectively, of the type graph of a semantic model. If e is an arrow from the type u to the type v, and if e′ is a query arrow from the query type u′ to the query type v′, and if e′ is a copy of e, then it is required that u′ is a copy of u, and that v′ is a copy of v. In this sense, the function mapping each copy of each type or arrow to the original type or arrow, respectively, is called a homomorphism from the query graph to the type graph. A query arrow is either a query attribute or a query specialization, if it is a copy of an attribute or a specialization, respectively. Query graphs can be drawn in the same way as type graphs. One can associate a selection and/or a negation to any query type. One can use a selection, for example, to specify that a certain constant value is required for a certain copy of a scalar type; this is called a constant selection. Another example of a selection is a wildcard selection, selecting any object of the type considered. Certain copies of scalar types (without negation) can be defined to be result types. As will be seen below, these result types are used to obtain a result of the query by projection. When compared to SQL queries, which are necessarily textual and linearly ordered, graph-based queries as considered here provide descriptions of queries with less syntactic noise.

Before considering the results of queries, it is useful to illustrate the approach with three examples. We refer here to FIG. 6. FIG. 6(a) is the type graph which appeared in FIG. 3(a) and was explained earlier on. FIG. 6(b) shows the query graph for the query: get supplier names for suppliers who supply part P₁. The selection of supplier P₁ is indicated below query type P. The projection on the name of the query type S is not shown. FIG. 6(c) shows the query graph for the query: get supplier names for suppliers who supply at least one part not supplied by supplier S₂. This query graph contains one type copy for the type P and two type copies for each of the types SP and S. The selection of supplier S₂ is indicated below query type S′. The sign at the top right hand corner of the query type SP′ indicates negation.

Referring to FIG. 7, it can be seen that this query asks for all vehicles that are not aeroplanes.

The result of a query is defined in terms of an intermediate result that consists of “tuples of objects”. Each tuple of objects contains, for each query type v′ without a negation, an object that is an instance of the type v of which v′ is a copy. Suppose that u′ and v′ are query types without negation; then a query attribute from a query type u′ to query type v′, which is a copy of the attribute e from u to v indicates that each tuple of objects contains objects for u′ and v′ in accordance with the attribute e between u and v. A query specialization between two query types u′ and v′, both without negation, indicates that each tuple of objects should contain the same object for u′ and v′. Each tuple of objects is required to satisfy the conditions expressed by the selections of the query. Query arrows are also referred to herein as internal joins, as they are derived from (i.e. copied from) the original semantic model. (Most of the joins of a query as formulated in a classical relational way become query arrows.) If a query type v′ appears with negation (and a selection), then it is a requirement that, for each tuple of objects, no object x exists (satisfying the selection) that is an instance of the type v of which v′ is a copy, such that x is related to the tuple's objects in accordance with the query arrows connected to v′. In addition to selections, negations, and projection, a query graph can also be extended with external joins, that is, joins not derived from the semantic model. For an external join, between two query types u′ and v′, it is required that the types u and v of which u′ and v′ are copies, respectively, should be connectable, possibly indirectly, by specializations in the type graph; in addition, it is required that each tuple of objects in the intermediate result should contain the same object for u′ and v′. For the result of a query, a projection is made: for each tuple of objects in the intermediate result only the values of the result types are kept. In this way, the result of a basic query is defined as a set of tuples consisting of values in the domains of the original semantic model.

For several applications of graphical queries, it is of interest to enable the user to define an ordering of the result. The result set of a basic query can be ordered by defining an order for one or more result types of the query (a numerical or alphabetical order, for example), and by lexicographically combining these orders in some order. Below we shall consider the application of queries to generate form-based applications and textual reports. In such applications, it is also of interest to order the intermediate result of a query. Such an ordering can be defined by the user in a similar way, by lexicographically combining orderings for certain copies of scalar types without negation.

By combining basic queries in a natural way into union queries and composite queries a rich query formalism is obtained. Before giving details about query composition in general, we give an example.

Referring now to FIG. 8, we illustrate that an analogue of relational completeness is obtained. A composite query presenting names of suppliers supplying all parts is presented. FIG. 8(a) is again the type graph which appeared in FIG. 3(a) and was explained earlier on. As an intermediate result, a basic query with negation determines pairs, consisting of a supplier and a part not supplied by this supplier (FIG. 8(b)). This basic query is denoted by Q: it's result contains the names of suppliers that do not supply at least one part. In the composite query FIG. 8(c) the query Q is used as a query type with a scalar type name. The result of the composite query is the set of suppliers not appearing in the result of Q. The composite query also uses a type copy S″ of S. There is an external join, depicted by a middle-middle connection between vertical sides to equate names between S″ and Q.

Composite queries

In order to define query composition, we first have to define union queries. Given a semantic model M, a basic union query on M is a finite set of basic queries on M with equal sets of result types. In addition, any of these result types is assumed to be a copy of the same scalar type in the given semantic model, for each query contained in the basic union query. The result of a basic union query is simply defined to be the union of the results of the contained queries.

In order to define query composition, we also need to define result models and result instances. The result model of a basic query on a semantic model M is another semantic model, consisting of one composite type, with scalar attributes to describe the value for each result type of the basic query. The domain of each scalar type of this result model is the domain of the original scalar type of the given semantic model M. It is clear that the result model of a basic query is essentially a relational table, and that the result of a basic query defines an instance of the result model, which will be called a result instance. The result model of a basic union query on M is the result model of any query contained in the basic union query. The result instance of a basic union query is the combination of the result instances of the queries contained in the basic union query, assuming that these instances do not share objects. This assumption can be justified as follows: it is clear that an object of an instance of a semantic model can always be replaced by another object, not yet in the instance, if the values and the references of the replaced object are given to the new object, and if references to the replaced object are replaced by references to the new object.

We can now define query composition, in a recursive way. Given a semantic model M, and a finite number of queries Q₁, . . . , Qₙ on M (for example, basic queries or basic union queries), we can define a new semantic model M′, containing the original semantic model M as well as the result models of the queries Q₁, . . . Qₙ. In the semantic model M′, composite types coming from different models become distinct types, and scalar types that arise from the same scalar type in M become identified in M′. Assuming that the instance of M and the result instances of Q₁, . . . , Qₙ do not share objects (this assumption has already been justified), these instances can be combined to form an instance of M′. A query on M is defined to be a basic query or a basic union query on M′. In this recursive definition, it is assumed that the directed graph of query dependencies (queries being defined in terms of other queries) is finite and does not have cycles (that is, there is no cyclic chain of dependencies). A query on M that is not a basic query on M is called a composite query on M. It is clear that for each query on M a result model and result instance is defined. In this definition of query composition, different types arising from the same scalar type v of M are combined into the same type v of M′, so as to be able to use external joins in a meaningful way.

As will be evident from the above, there has been described a convenient means by which graphical queries may be composed, in the context of objects, types of objects, relationships between objects, and specialization relationships between types of objects. When relational schemas and queries are transformed to this framework, it can be seen that a relationally complete approach is obtained. The construction of relatively complex queries may be achieved with practice in a straight forward, easy to use manner.

Query user interfaces

It is clear from the preceding sections that semantic models and queries can be defined using directed graphs, and hence using direct manipulation. A graphical notation for semantic models can be applied when using graphical relational tools such as Access for implementation, and is convenient in keeping track of types and arrows, especially for more complicated semantic models and queries. Since inheritance is dealt with by means of specializations, it can be handled in a convenient, well integrated way. One can, in fact, suggest a further improvement to facilitate the specification of queries even for semantic models without specializations. As was already noted, most of the joins of a query in a classical relational form become internal joins, that is, attributes in the query graph, connected to the type graph via the homomorphism of the query. Hence a user interface for the construction of queries might enable the user to select certain sub-graphs of the type graph for automatic redrawing in the query graph. In other words, the user might construct a query graph by means of copy and paste operations starting from the type graph. The user would then only need to explicitly draw connections for the external joins. In the development of queries, it is attractive to have insight in and to make use of the predefined relationships between types, in this way. Thus, it is not necessary to repeat the specification of this information on relationships frequently in the formulation of queries.

Updatable views, retractions

We now turn to the exploitation of graphical queries in data management systems. An updatable view for a given semantic model can be realised by another semantic model, called a view model, whose types and arrows are copies of the types and arrows of the original semantic model, in the same way as with the query graphs described in a preceding section. An instance of the original semantic model can be used to define an instance of the view model. We call the latter instance a retraction. Modifications to the original data can be done via the retracted instance of the view model. This can be used to define form-based applications, allowing inspection and modification of information from an alternative point of view. These applications can be automatically generated on the basis of information that can easily be specified in a graphical way.

There is an interesting application of this procedure in connection with the World Wide Web. It is customary to present information on the Web in a fixed, serialized (textual) way, using XML or HTML. In many cases it is also possible, however, to present an overview of the structure of certain information, using a semantic model, displayed in a graphical way, and to allow a user to define a specially desired view, using a graphical query, and to present the result using specially generated XML. In this way, a user could define his own form-based application for inspection, and even for modification of the information.

Before describing the proposed procedure for inspection and modification of data in general, an example will now be given, referring to FIG. 9, in which, given simply, graphically specified information, an interactive application is automatically provided for inspection and modification of information concerned with music. The purpose of the semantic model is to record information on music performances and performers, based on pre-definable genre information. The main types of the semantic model can be explained as follows. Each “piece” of music is described using a “composer” and a “genre”. In order to perform the piece, certain “roles” are required, such as pianist, orchestra, and conductor, for a piano concerto. The default roles used for pieces of a certain genre can be described by means of the type “role-genre”. If these default roles are not suitable for a piece of a certain genre then the roles used for this piece should instead be described with the type “role-piece”. These types role-genre and role-piece are disjoint specializations of the type “role-use”, which has an attribute of type role. In this way, the type role-genre can essentially be viewed as an m:n relationship between the types role and genre, for which exceptions can be specified using the type role-piece. To give a concrete example, a piano concerto with two pianos can be described as a piece with four role-piece objects (orchestra, conductor, pianist, pianist), to be taken instead of the three role-genre objects of the piano concerto genre. There may be several performances for a piece. Each performance belongs to a certain recording “label”. The “participations” of a performance describe the “performers” who realise the roles used for the piece performed. A static constraint is that, for each role-use object of a piece (or it's genre), there is at most one participation for each performance of the piece.

Supposing that it is wished to create an interactive form-based application for inspection and modification of information on piano concerto performances. As indicated in FIG. 10, a form describing a piano concerto performance lists the composer, the piece, and the place, time and label of recording. There is a sub-form for the pianist(s). There are sub-forms for the conductor and the orchestra, both of which are expected to contain only one object.

This application can be constructed in a graphical way by using Microsoft Access, for example: utilising Access one would make and integrate four forms, based on four separate (graphically defined) queries. We describe a simpler user interface for developing such an interactive application, based on just one graph-based query. This query is shown in FIG. 11.

In order to select conductor, pianist and orchestra, the query graph contains three copies of each of the types participation, role-use, role and performer. The selections pianist, conductor, orchestra and piano concerto are displayed immediately below the appropriate query types. In order to automatically generate the form-based application, this graph-based query only needs to be extended with the assignment of genre as a specially selected query type, with the assignment of the query type performance as a form type (indicated by an * in FIG. 11), and with the indication of the following query types as sub-form types (indicated by the symbol˜in FIG. 11): composer, piece, label, perf 1, perf 2, perf 3. The sub form types place and time are not displayed in FIG. 11.

FIG. 11 defines a semantic model, the query model, which is an example of the view models already mentioned. An instance of the original semantic model of FIG. 9 can be viewed as defining an instance of the query model, called a retraction. When a type, of which an object is an instance, has different copies in the query graph, a retraction contains different copies of this object, one object copy for each type copy. Relationships between objects, and values of objects, are taken over in a natural way by the object copies. When one takes into account the selections of the query, instead of the retraction, one obtains a smaller instance of the query model called the retracted sub-instance generated by the query. Even this retracted sub-instance may contain different copies of the same object. For example, if the instance of the semantic model of FIG. 9 contains the performance of the Mozart Piano Concerto played and conducted by Ashkenazy, then the retracted sub-instance contains two Ashkenazy objects, one for the role pianist and one for the role conductor.

In this way, a view model, defined using a graphical query, and its retracted sub-instance, are useful to define an interactive, form-based application. It should be noted that a view model and its retracted sub-instance can also be used to generate a textual report, for example represented using XML. For the automatic generation of form-based applications as well as textual reports, it is preferred to enable a user to specify orderings of information items. A natural way to do this is by defining an order on the set of tuples of objects in the intermediate result of a query, in the way already described.

Basic update operations

We turn to a more general description of updatable views. To this end, we first consider the kinds of modifications of instances that should be considered. As a consequence of the definition of instance of a semantic model, it is clear that modifications of instances can be defined in terms of six basic update operations, defined as follows:
Value assignment: an object gets a new value for one or more scalar types that are connectable, in the given semantic model, via specializations; for each of these scalar types, the object gets the same new value.
Reference assignment: for an attribute e from a type v to a type w, and an object x that is an instance of v, the reference of x to an object that is an instance of w is changed.
Object creation: a new object is added to the instance, with a value for each scalar type of which it is an instance, and a reference for each attribute of each composite type of which it is an instance.
Object deletion: an object is deleted from the instance, together with its values for the scalar types of which it is an instance, and its references to other objects; this can be done only if there do not exist references to the object to be deleted.
Object type extension: an object becomes an instance of a type of which it was not yet an instance, and obtains a new value if this type is scalar, and obtains new references if this type has attributes.
Object type reduction: an object stops being an instance of a type, and loses its value for this type if this type is scalar, and its references for the attributes of this type if this type is composite; this can be done only if no reference exists to the object for an attribute pointing to the omitted type of the object.

Updates of information via retractions

We shall now describe details about the way in which view models, defined using graphical queries, enable updates on information. Suppose that a semantic model is given, as well as an instance of this semantic model, and a basic query on this semantic model. Recall that the basic query defines a homomorphism from its query graph to the type graph of the semantic model, which is a function mapping each type copy or arrow copy in the query graph to the original type or arrow in the type graph, respectively. The basic query defines another semantic model, called a view model. We shall assume that each scalar type of the view model is a copy of a scalar type of the original semantic model. The domain of a scalar type of the view model is taken to be the domain of the type of which it is a copy.

The given semantic model, instance, and basic query together define an instance of the view model, called a retraction, in the following way. Each object of the retraction is a copy of an object of the original instance. Suppose that x is an object of the original instance, that x is an instance of the set of types V in the given semantic model, and that V′ is the set of types in the view model that are copies of a type in V. Let W′ be a subset of V′ such that each pair of types in W′ can be connected, possibly indirectly, via specializations in the view model, and such that W′ cannot be extended to include other types of V′, without losing this property. Then the retraction contains an object copy x′ of x, in such a way that x′ is an instance of precisely the types in W′. The retraction contains different objects for different sets W′ obtained in this way, and these are all the objects in the retraction. If v′ is a scalar type of the view model, v′ is a copy of v, x is an instance of v, x′ is a copy of x, and x′ is an instance of v′, then the value of x′ for v′ is taken to be the value of x for v. If v is a composite type of the original semantic model, e is an attribute of v, v′ is a copy of v, e′ is a copy of e and an attribute of v′, x is an instance of v, x′ is a copy of x and an instance of v′, then the object referred to by x′ in accordance with e′, is required to be a copy of the object referred to by x in accordance with e. It is not difficult to see that this last requirement can be realized in exactly one way.

This completes the definition of a retraction. We also need to consider certain sub-instances of retractions. A sub-instance of an instance of a semantic model is a second instance that consists of a subset of the objects of the first instance, together with the original values and references of these objects. The retracted sub-instance generated by a basic query is defined to contain, first of all, those objects that satisfy the selections of query types that were assigned to be specially selected, as in the piano concertos example already discussed. In addition, a retracted sub-instance contains other objects as well: an object x of the retraction is part of the retracted sub-instance if it refers, possibly indirectly (that is, possibly via more than one attribute), to an object that satisfies one of the already mentioned special query type selections, and if x does not refer, possibly indirectly, to objects that do not satisfy a query selection. The definition of a retracted sub-instance is made complete by including also those objects needed to incorporate the references of each object included.

Given this discussion of retractions and retracted sub-instances, it remains to describe modification of instances via the graph-based query. For each of the basic update operations that were distinguished, performed on a retraction or on a retracted sub-instance, it is necessary to arrive at consistent incorporation in the original instance and in the (remainder of the) retraction. It is important to note that changes involving an object x′ in the retraction are always reflected in the object x of which it is a copy, and also in the other object copies of x. The definition of a retraction shows that, for each of the basic update operations, modification of an instance can be incorporated in a retraction, and vice versa. The main point is that, given an instance, values and references in a retraction are always uniquely defined. For example, suppose that x′ is a copy of an object x, with a reference to y′ for attribute e′, while y′ is a copy of y and e′ is a copy of e. Suppose that z′ is a copy of the object z, and that the reference of x′ (for e′) to y′ is changed to z′. Then the reference of x (for e) is changed to z, and, in turn, each corresponding reference of other copies of x is changed in the unique way maintaining the properties of a retraction. For the incorporation of modifications of a retracted sub-instance, additional assumptions have to be made for three of the basic update operations, namely reference assignment, object creation, and object type extension. For reference assignment on a retracted sub-instance, for example, it is assumed that the resulting reference updates in the retraction do not violate the assumptions on a retracted sub-instance, that is, are in line with the selections of the query. Object creation and object type extension are subject to an analogous assumption.

Form-based applications

The example of FIGS. 9-11 illustrates that a possibly complex form-based application, allowing inspection and modification of data, can be specified in terms of just one basic query on a semantic model. Using the material in the preceding sections on updates, we can now describe this procedure in general. Suppose that a semantic model and a basic query on this model is given, and that each scalar type of the basic query is a copy of a scalar type of the semantic model. Suppose that the basic query does not contain negations, and that each selection is a constant selection or a wildcard selection. Suppose, in addition, that a certain query type is chosen as the form type, that certain other query types are chosen as sub-form types, and that certain query types with selection are chosen as specially selected query types. In order to describe the relationships between these types more precisely, we have to consider certain paths in the query graph. In general, a path in the query graph from a type v′ to a type w′ may contain arrows (that is, attributes or specializations) in a forward or reverse manner. It is assumed that there is a path in the query graph, consisting of only forward attributes and forward specializations, from the form type to the specially selected query type. We consider three kinds of sub-form types. Denoting the form type by v′ and a sub-form type by w′, it is assumed that either there is a path in the query graph from v′ to w′ using forward attributes and forward or reverse specializations, or there is an attribute from w′ to v′, or there is a type u′ and an attribute from u′ to w′ and an attribute from u′ to v′. It is clear that the query graph of FIG. 11 satisfies these conditions. It is also clear that a form can be automatically generated, which can display an object that is an instance of the form type v′, with sub-forms for the sub-form types w′ displaying objects related to the first object. More precisely, the system of form and sub-forms displays tuples of objects in the retracted sub-instance of the query. It is clear that for each object displayed in the form, at most one object is displayed in a sub-form of the first type, and any number of objects might be displayed in sub-forms of the second and third types. A form or a sub-form displays values for scalar types of the form type or sub-form type, respectively. If an object displayed in a form or sub-form is also an instance of specializations of the corresponding form type or sub-form type, respectively, then values can also be displayed for scalar attributes of these specializations. Modification to data can be done in such a form-based application, using the procedures described in the preceding sections. It should be noted that object creation for a sub-form type w′ of the third kind, requires the creation of an additional object of the already mentioned type u′ connecting w′ with the form type v′. In this case, it is natural to maintain the situation that there is precisely one such “connecting object” that is an instance of u′, for each pair consisting of an object that is an instance of v′ and an object that is an instance of w′.

A generalization of the setup thus described is obtained by noting that for a sub-form type one may also assign sub-sub-form types. Such sub-sub-form types are assumed to stand in the same relation to the sub-form type as was assumed above for the relationship between sub-form types and the form type. This allows the construction of a form-based application consisting of a main form, containing sub-forms, where sub-forms may also contain sub-forms. This can be extended even further, to construct, using just one basic query, form-based applications with arbitrarily deep nesting of sub-forms, and presenting and managing a special view on an instance of a semantic model.

It should be noted that when only inspection of data through a form-based application is desired, and no modification, then it is not necessary to make the assumption that the basic query does not contain negations. In this case it should be assumed, however, that the form types and sub-forms types are query types without negation.

It should also be noted that in these considerations of form-based applications we do not incorporate questions of precise layout of items on forms and sub-forms. The invention concerns the automatic generation of structure and functionality of form-based applications, based on easily specifiable graphical information. It remains necessary to design a specific layout for such a form-based application. Given structure and functionality of a form-based application, this can be done, for example, using easy-to-use facilities such as made available by the Access system. A similar remark pertains to the textual reports discussed in the next section.

Textual reports

It has already been mentioned that a view model (constructed using a graph-based query) can also be used to automatically generate a textual report. This can proceed in a way analogous to the automatic generation of form-based applications discussed in the preceding section. Suppose that a basic query on a semantic model is given, and that each scalar type of the query is a copy of a scalar type of the semantic model. In addition, a certain query type without negation is assumed to be assigned as the report type, and certain other query types without negation may be assigned as sub-report types. Just as with form-based applications, it is possible to assign certain query types with selection as specially selected query types. The report type and the sub-report types are assumed to satisfy the same relationship as assumed in the preceding section for the form-type and the sub-form types. The specification of a textual report can be made more complete by specifying certain orderings. For example, an ordering can be specified on the sub-report types. For the report type and for each sub-report type, an ordering can be specified on its scalar query types. An ordering can also be specified on the tuples of objects in a retracted sub-instance of the view model defined by the basic query: this can be done conveniently in terms of scalar types without negation. These specifications can be used to automatically generate a textual report presenting the retracted sub-instance of the view model defined by the basic query. For each object that is an instance of the report type, the report presents the values for all scalar query types of the report type, and presents a sub-report for all sub-report types. Each sub-report presents values for all scalar attributes of the sub-report type. Just as with a form-based application, when an object displayed in a report or sub-report is also an instance of specializations of the corresponding report type or sub-report type, then values can also be displayed for scalar attributes of these specializations. Such a nested textual report could be realized using the standard Web language XML. It should be noted that just as with form-based applications, deeper nesting of sub-reports can be realized in an analogous way. In this way, one can construct a possibly complicated, textual report, on the basis of just one graphical query.

It should be noted that what we call a textual report is by no means restricted to the presentation of text. As has already been mentioned, scalar types may involve complicated graphical or multimedia objects. Instead of the more standard term textual report, the term “information serialization” might describe the intended meaning in a better way.

Semantic models: expressivity

Graph-based queries, and their applications, as considered here, are based on the definition of semantic models and their instances. In the arrangements of the present invention, the number of modelling primitives is minimised, but in spite of this the formalism of semantic models has a large expressive power. Many data modelling constructs can be form alised in terms of semantic models. For instance, a relational schema can be viewed as a semantic model in which each arrow is a scalar attribute; the standard instances are obtained if one restricts the general, object-oriented instances to ‘value-based instances’, which do not contain two objects, of a composite type, with the same value for each scalar attribute of the composite type. In terms of semantic models, one might say that the relational model supports one “level” of attributes. A schema according to the well known entity-relationship model can be expressed as a semantic model with two “levels” of attributes: entities can be described with types, each of whose attributes is scalar, and relationships can be viewed as types with attributes pointing to such “entity types”. It is clear that 1:n-relationships can subsequently be replaced by additional attributes. Isa relationships can be handled using specializations.

In the foregoing text, there has been described a method by which semantic modelling may be implemented to enable user friendly interfacing with data. This data modelling formalism, based on semantic models and object-oriented instances, can be viewed as an extension of the relational model, and shares several important strengths with the relational model: data independence, view independence, and simplicity of basic primitives. It can be mathematically formalized in terms of sets, functions, and graphs. Several aims are achieved that are not realised with the relational model, namely: information modellers can express semantics of data, in terms of objects, types, relationships between objects, and specializations (the basic primitives were chosen so as to achieve this in the simplest possible way); graphical query user interfaces can directly be considered in terms of these object-oriented primitives, by means of graph-based queries; these graph-based queries can be used to realize an easy to use way of constructing updatable views in the form of interactive database applications, or of constructing possibly complex, textual reports.

Inheritance is realised without restrictive assumptions on the structure of type graphs. General semantic models and object-oriented instances can be implemented relatively easily in terms of the relational model, or in the form of web data (XML). A succinct abstract description of queries, based on semantic models, was obtained: a semantic model is expressed using a type graph, and a query is expressed using a query graph with a homomorphism from this query graph towards the type graph. Restricted to the relational model, the graph-based queries defined here form a relationally complete query formalism. Easy non-procedural access, by a graphical user interface, can be extended to general semantic models and object-oriented instances. User interfaces for setting up queries can be simplified by supporting the marking of sub-graphs of the type graph for incorporation into the query graph, combined with the facility to draw external joins (that is, joins not derived from the type graph). A significant class of interactive database applications can be developed in an easy, graphical way, as updatable views defined in terms query graphs. A form-based application, or a textual report, with possibly complex nesting of sub-forms (or sub-reports, respectively) of various different, regular structures, can be constructed using just one graphical query.

Whilst in the above description, reference has been made to the Microsoft Access system, it will of course be appreciated that the teachings of the invention are not limited to such a system and that the principles described are directly applicable to database applications in general, and also to the management of information not necessarily represented in databases but on the World Wide Web, for example.

[Booch et al 1999]

Booch, G., Rumbaugh, J., and Jacobson, I. 1999. The Unified Modeling Language User Guide. Addison-Wesley, Reading, Mass.

[Codd 1970]

Codd, E. F. 1970. A relational model of data for large shared data banks. Comm. ACM 13, 6, 377-387.

[Codd 1982]

Codd, E. F. 1982. Relational databases: a practical foundation for productivity. Comm. ACM 25, 2, 102-117. [Ter Bekke 1992]

Ter Bekke, J. H. 1992. Semantic Data Modelling. Prentice-Hall, N.Y.

[Tsichritzis and Klug 1978]

Tsichritzis, D. C., and Klug, A., Eds. 1978. The ANSI/X3/SPARC DBMS Framework: Report of the Study Group on Data Base Management Systems. Information Systems 3, 173-191.

1. A graphical user interface for a data management system, the user interface comprising:
means for defining in a graphical format a semantic model defining the structure of data within a database; and
means for using said semantic model to define a database query, the interface being characterised in that the semantic model comprises a type graph comprising types of objects, and attributes and specializations linking types to one another to define relationships between said types.

2. The graphical user interface of claim 1, wherein said type graph is represented according to a pre-defined graphical notation wherein types are represented in a particular manner and associated with a type name and attributes and specializations are represented as linking types to one another.

3. The graphical user interface of claim 1, wherein said means for using said semantic model comprises using said type graph to define a query graph.

4. The graphical user interface of claim 3, wherein said query graph comprises query types, query attributes and query specializations being respectively derived from types, attributes and specializations of said type graph.

5. The graphical user interface of claim 4, wherein a query type is associable with a selection and/or a negation.

6. The graphical user interface of claim 4, wherein a query graph is constructable by means of copy and paste operations carried out on a type graph.

7. The graphical user interface of claim 4, wherein a query graph further comprises reference to at least one other query graph.

8. The graphical user interface of claim 4, wherein an interactive, form-based application is realisable by construction of a query graph.

9. The graphical user interface of claim 8, wherein the form-based application comprises a form having nested sub-forms.

10. The graphical user interface of claim 8, wherein modification of data is achievable using the form-based application.

11. The graphical user interface of claim 4, wherein a textual report is realisable by construction of a query graph.

12. The graphical user interface of claim 11, wherein the textual report contains nested sub-reports of various different, regular structures.

13. A method of presenting and managing data graphical queries in a data management system comprising:
defining in a graphical format a semantic model defining the structure of data within a database; and
using said semantic model to define a database query, the method being characterised in that the semantic model comprises a type graph comprising types of objects, and attributes and specializations linking types to one another to define relationships between said types.

14. The method of claim 13, wherein said type graph is represented according to a pre-defined graphical notation and comprises types being represented in a particular manner and associated with a type name and attributes and specializations being represented as linking types to one another.

15. The method of claim 13, wherein using said semantic model comprises using said type graph to define a query graph.

16. The method of claim 15, wherein said query graph comprises query types, query attributes and query specializations being respectively derived from types, attributes and specializations of said type graph.

17. The method of claim 16, wherein a query type is associable with a selection and/or a negation.

18. The method of claim 16, wherein a query graph is constructable by means of copy and paste operations carried out on a type graph.

19. The method of claim 16, wherein a query graph further comprises reference to one or more other query graphs.

20. The method of claim 16, wherein an interactive, form-based application is realisable by construction of a query graph.

21. The method of claim 20, wherein the form-based application comprises a form having nested sub-forms.

22. The method of claim 20, wherein modification of data is achievable using the form-based application.

23. The method of claim 16, wherein a textual report is realisable by construction of a query graph.

24. The method of claim 23, wherein the textual report, contains nested sub-reports of various different, regular structures.

25. A data management system comprising a graphical user interface as claimed in claim 1.

26. A computer program product enabling a programmable device when executing said computer program product to function as a data management system as claimed in claim 25.
